(19) United States
(12) Patent Application Publication
(10) Pub. No.: US 2020/0178793 A1
(43) Pub. Date: Jun. 11, 2020

(54) Refraction Measurement of the Human Eye with a Reverse Wavefront Sensor

(71) Applicant: EyeQue Inc., Newark, CA (US)

(72) Inventor: Noam Sapiens, Newark, CA (US)

(21) Appl. No.: 16/705,999

(22) Filed: Dec. 6, 2019

Related U.S. Application Data
(60) Provisional application No. 62/776,041, filed on Dec. 6, 2018.

(51) Int. Cl. A61B 3/10; A61B 3/113; G01J 9/02

(52) U.S. Cl. CPC A61B 3/1015; G01J 2009/002; G01J 9/0246; A61B 3/113

(57) ABSTRACT

A wavefront sensor measures the phase distribution of a beam of light perpendicular to its axis of propagation. The Shack-Hartmann (S-H) wavefront sensor is based on segmentation of the incident light beam into small, spatially distributed, parts. Each of these parts is then incident on a lens, and the deviation of the focal spot from the lens optical axis is measured in two dimensions, usually by a camera or detector array. An array of lenses is used to characterize the wavefront of the entire beam.

### CROSS-REFERENCE TO RELATED APPLICATIONS

This application claims the priority of and benefit of U.S. provisional patent application 62/776,041 filed on Dec. 6, 2018, the contents of which are incorporated herein by referenced as if restated herein.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### BACKGROUND OF THE INVENTION

### (1) Field of the Invention

The invention generally relates to vision measurement systems. More particularly, the invention relates to means and methods of using a reverse wavef front sensor to measure errors in an optical system.

### (2) Description of the Related Art

The known related art fails to anticipate or disclose the principles of the present invention.

In the related art, general methods and systems of measuring optical characteristics are known and include:

Stokes G. G., “On a mode of measuring the astigmatism of a defective eye”, Mathematical and Physical paper, Cambridge University Press, 2, 172-5, 1883.

Arines J., Acosta E., “Adaptive astigmatism-correction device for eyepieces”, Opt. and Vis. Sci. 88(12), 2011.

### GENERAL BACKGROUND

Vision is arguably the most important of the senses. The human eye and its direct connection to the human brain is an extremely advanced optical system. Light from the environment goes through the eye optical train comprised of the cornea, the pupil, and the lens and focuses to create an image on the retina. As all optical systems, light propagation through the eye optics is subject to aberrations. The most common forms of aberrations in the eye are defocus and astigmatism. These low order aberrations are the cause of the most common refractive eye conditions myopia (nearsightedness) and hyperopia (farsightedness). Higher order aberrations are also present and can be described most conveniently by the Zernike polynomials. These usually have a lower effect on visual function. The eye, like any other organ in the human body, may suffer from various diseases and disorders, the most prominent today are: cataract, AMD, glaucoma, diabetic retinopathy, dry eye.

Ophthalmic measurements are critical for eye health and proper vision. Those ophthalmic measurements could be sectioned into objective and subjective types. Objective types measurements give a metric of a physiological, physical (e.g. mechanical or optical), biological or functional without the need for input from the measured individual (patient, subject, user or consumer). Examples of objective tests include but are not limited to OCT (optical coherent tomography used to image a 3 dimensional and cross sections of the eye), scanning laser ophthalmoscope (SLO, used for spectral imaging of the retina), fundus image (used to present an image of the retina), auto-refractor (used for refraction measurement), keratometer (used for providing a profile of the cornea), tonometer (used to measure the IOP—intra ocular pressure). Subjective measurements give a metric with relation to the individual input. That is, they provide parameters that also take into consideration the brain functions, perception and cognitive abilities of the individual. Examples of subjective tests include but are not limited to visual acuity test, contrast sensitivity test, phoropter refraction test, color vision test, visual field test, and the EyeQue PVT and Insight.

Today, both objective and subjective eye exams (measurements) are done by an ophthalmologist or an optometrist. The process usually involves the patient needing to schedule an appointment, wait for the appointment, travel to the appointment location (e.g, office or clinic), wait in line, perform multiple tests using various tools and potentially moving between different technicians and different eye doctors. The prolonged wait times both for the appointment as well as in line at the appointment location, along with the hassle of performing the tests with different professionals and the duration of those tests might seem daunting to many patients. Furthermore, the shear effort associated with the process and even the requirement of remembering to start the process to begin with might deter patients from going through with a traditional examination.

Moreover, currently about 2.5 billion people do not have access to eye and vision care at all. The cost of eye exams could be considered quite significant especially in some places in the world. This poses a hindrance to the availability of eye care in third world countries for example. The cost, time consumption and perceived hassle also makes it at times prohibitive to have repeated eye exams, especially at the desired frequency. Those might be necessary in special cases (for example after refractive surgery or cataract surgery where repeated measurements should be performed to track the progress of the patient's status over time and the success of the surgery. Additionally, even under normal circumstances, measurements at a doctor's office only represent a single point in time. The situation under which the measurements were made might not be optimal or do not fully represent the patient's characteristics. The patient might have been tired, stressed or agitated (a doctor's visit might be quite stressful in and of itself but could also being run from test to test and being posed with questions and options elevate the patient's level of stress) or was just in a bad mood. Even the state of mind of the doctor themselves might influence the way the measurement is performed. Beyond all that, the time of day and other environmental conditions (whether direct e.g. lighting conditions or indirect e.g. temperature) could also affect the measurement and provide incomplete or false information.

The availability of information (including specifically medical information) on the Internet, the increased awareness of people for preventive medicine, and the emergence of tele-medicine leads to many taking control of their own health. Devices for screening, monitoring and tracking medical conditions are quite pervasive in today's world, for example blood pressure measurement devices, and blood sugar monitors. The technological advancements allow for people to be more independent in diagnosis, prevention and tracking of various health conditions. Furthermore, many prefer to perform these activities in the comfort of their homes without the need for appointments or other time-consuming activities. In case of an anomaly, they would call or email their physicians to consult for the appropriate course of action.

The advancement of technologies effectively makes computers with screens and cameras ubiquitous in the form of laptops, tablets and smartphones. Therefore, enabling many people to have a device already capable of computing displaying and recording information.

All this brings the need for a series of devices that will enable users to perform ophthalmic measurements at home, by themselves, in a timely and cost-effective manner. It should be clear that the quality of these measurements and their accuracy and precision should meet or exceed the standards of today's measurement methods.

This vision could be further enhanced by use of cloud-based data and analytics that enables complete access to the entire history of a patient exams, tests and measurements. Moreover, the use of artificial intelligence (AI) will enable diagnosis based on machine learning and big data. This could be done by means of data mining, neural network decision making and pattern detection and recognition, as some examples of the AI capabilities.

To summarize, the vision for eye care in the not so far future will look like:

A complete solution for eye and vision care for consumers and doctors.

Remote, self-administered battery of tests for both disease and functional measurements are enabled by technology and devices

AI is used for analysis, tracking and reporting. Enhanced by big data correlations and insights

In simple terms, as an example: A person sits on their couch at the comfort of their home, uses a device to do various measurements, that data is uploaded to an AI for analysis. The AI will let the person know the results and notify the doctor. The AI will initiate alerts for the person and doctor in necessary cases. The person will not need to get up unless a serious issue occurs (i.e. surgery). All other issues will be dealt with remotely (e.g. email/phone/video conference with the doctor, order glasses and have them delivered to the home, direct delivery of doctor prescribed medications).

Despite the apparent approach of “direct to consumer”, the methodologies could easily be implemented for a more enterprise like model. One example of such implementation will have a hierarchical structure in which an entity such as a hospital, association, or a medical insurance company provides the ability for the doctors to provide their patients with such devices and capabilities. The devices are all connected through the user accounts to the cloud and the measurements are streamed directly into the users' accounts (and potentially their medical records). Those accounts could be attached to one or more doctors and can also be transferred and shared.

### BRIEF SUMMARY OF THE INVENTION

The present invention overcomes shortfalls in the related art by presenting an unobvious and unique combination and configuration of methods and components to create an apparatus that may be used to find refraction properties for consumers.

In a disclosed embodiment, a wavefront sensor measures the phase distribution of a beam of light perpendicular to its axis of propagation. The Shack-Hartmann (S-H) wavefront sensor is based on segmentation of the incident light beam into small, spatially distributed, parts. Each of these parts is then incident on a lens, and the deviation of the focal spot from the lens optical axis is measured in two dimensions, usually by a camera or detector array. An array of lenses is used to characterize the wavefront of the entire beam. FIG. 4 presents a schematic representation of the S-H wavefront sensor principle.

A beam of light is incident upon a lenslet array that is aligned with a pixelated detector (e.g. CCD or CMOS camera), such that each lenslet optical axis is set at a single central pixel or a cross-section of pixels. If the beam of light has a uniform wavefront (e.g. plane wave), then the focal points from each lenslet will coincide with each lenslet respective optical axis equivalent on the detector array. As a distorted/aberrated wavefront is incident upon the lenslet array, the angle of incidence on each lenslet is different and produces a spot that its focus is offset from the individual lenslet optical axis. This deviation is related to the angle of incidence and therefore the local phase of the beam. Mapping the deviation from all the lenslets allows for data processing, e.g. matching the given pattern to the Zernike polynomials. This in turn allows for characterization of the aberrations of the incident beam. The order of the Zernike polynomials (the type of aberrations) that can be calculated in this measurement is dependent upon the number of lenslets. The lenslet power and the number of pixels behind each lenslet dictates the range and resolution (namely, accuracy) of the measured wavefront phase.

As defocus and astigmatism are aberrations that are represented by the Zernike polynomials and thus have distinct features in that space, this type of measurement, if could be applied to measuring eye aberration will provide valuable results.

A reverse S-H wavefront sensor was perceived and invented. FIG. 5 shows a schematic representation of the invention concept.

Description of the optical aspect of the invention (an example implementation): An image is presented on a display. The display is segmented. Each segment has a lens in front of it. The lens is placed such that the display is at the lens focal plane. The entire lens array is followed by a de-magnifier (reverse beam expander), the purpose of which is to condense the light from all the segments to fit into the entrance pupil of the measured system.

These and other objects and advantages will be made apparent when considering the following detailed specification when taken in conjunction with the drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 describes a first set of tasks in obtaining eye care in the prior art

FIG. 2 describes a second set of tasks in obtaining eye care in the prior art

FIG. 3 depicts entity management in the prior art

FIG. 4 depicts a disclosed system of optical measurement

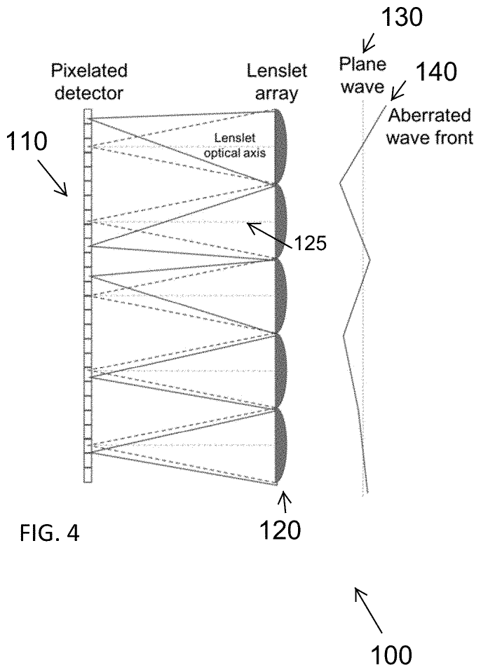

FIG. 5 depicts a disclosed system of optical measurement

FIG. 6 depicts images used in a disclosed system

FIG. 7 depicts a disclosed demagnification system

FIG. 8 depicts the use of a central image to create a 3D implementation for accommodation

### REFERENCE NUMBERS

100 a disclosed system in general

110 pixelated detector

120 lenslet array

130 plane wave

140 aberrated wave front

150 screen

155 baffles

160 3×3 lenslet array

170 de-magnifier

200 prior art methods of eye care

300 prior art entity management for eye care

400 images sometimes used for optical measurements

500 central image system to create a 3D implementation for accommodation

### DETAILED DESCRIPTION OF EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

Unless otherwise noted in this specification or in the claims, all of the terms used in the specification and the claims will have the meanings normally ascribed to these terms by workers in the art.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “above,” “below,” and words of similar import, when used in this application, shall refer to this application as a whole and not to any particular portions of this application.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not only the systems described herein. The various embodiments described herein can be combined to provide further embodiments. These and other changes can be made to the invention in light of the detailed description.

Any and all the above references and U.S. patents and applications are incorporated herein by reference. Aspects of the invention can be modified, if necessary, to employ the systems, functions and concepts of the various patents and applications described above to provide yet further embodiments of the invention.

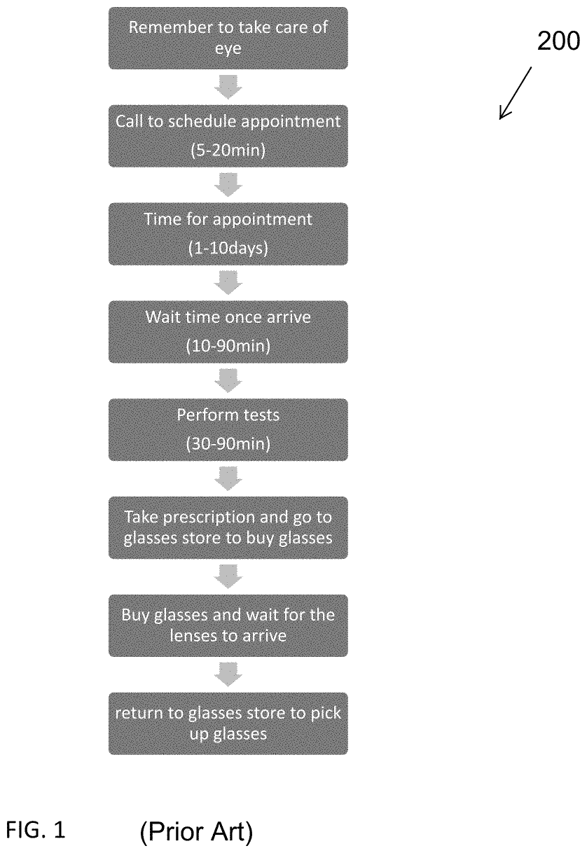

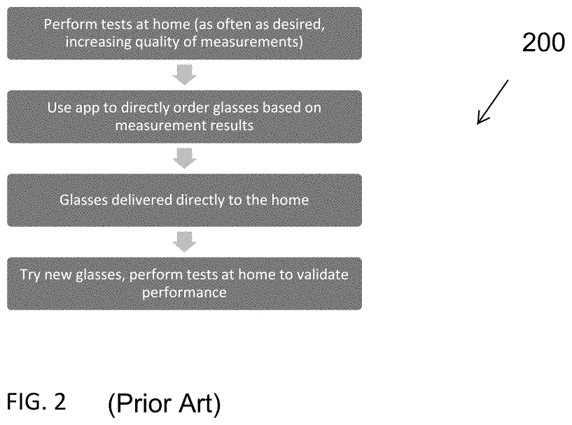

Referring to FIG. 1 and FIG. 2 a prior art system of eye care management 200 is described.

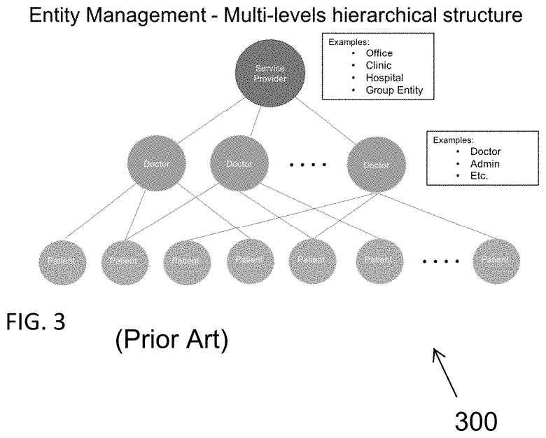

Referring to FIG. 3 a prior art system of entity management 300 for eye care is described.

Referring to 4 a disclosed system is depicted and may comprise a pixelated detector 110, a lenslet array, a lenslet optical axis 125, a plane wave 130 and an aberrated wave front 140.

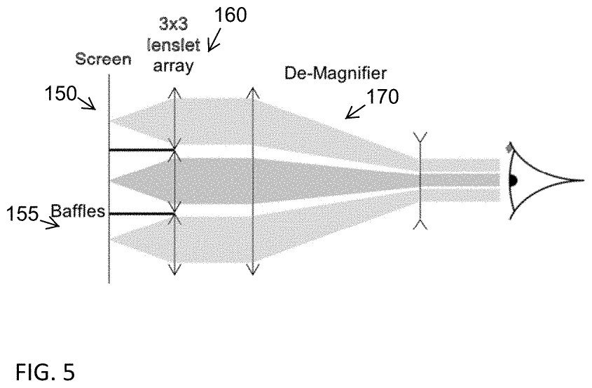

Referring to FIG. 5, a disclosed embodiment may include a screen 150, baffles 155, a 3×3 lenslet array 160 and a de-magnifier 170.

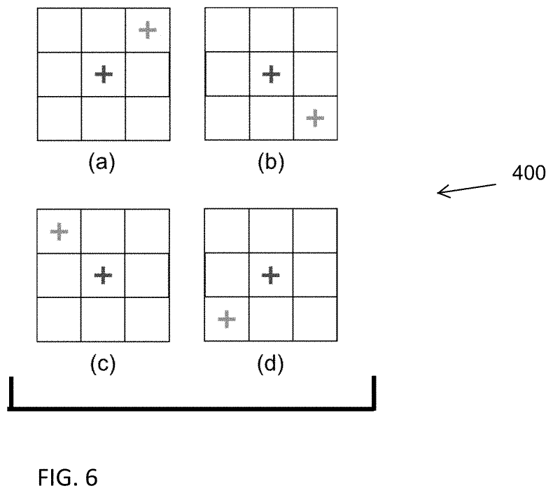

FIG. 6 depicts images 600 sometimes used for optical measurements.

The central segment of the display 600 presents an image of a red cross. In the subsequent steps of the measurement, this central image is static. The first step of the measurement includes a presentation of a green cross at one of the adjacent segments. The measured system detector is used to align the two crosses in two dimensions so that they overlap. The location of the green cross is recorded. The process is repeated for other segments of the display. Each step is independent from the other steps; thus, the green cross image is displayed on only one segment at a time. The collection of recorded locations is then used in the analysis to determine the Zernike profile of the measured system and could be used to determine the required correction of such system. Correction in this respect means providing a uniform wavefront.

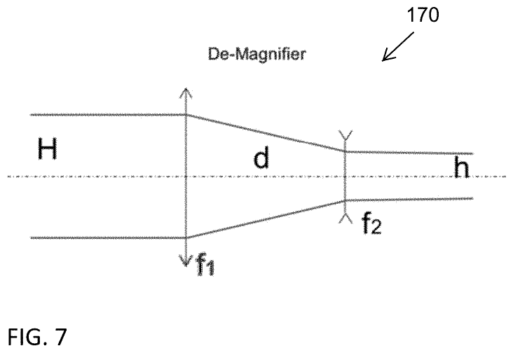

FIG. 7 depicts a de-magnifier 170. In one disclosed de-magnifier, the following equations should be met:

f_1+f_2=d

Magnification=h/H=f_2/f_1

The de-magnifier is comprised of a positive and a negative lens, the de-magnifier is comprised of two positive lenses, the de-magnifier is built to cover the entire pupil of the measured system, the de-magnifier is built to cover a portion of the pupil of the measured system. The de-magnifier is built to combine the beams from the individual lenslets. The de-magnifier is built to create a collimated beam space. The de-magnifier is used to improve the resolution of the device.

The image presented can be of a cross, a star, an Asterix, or any other image. The central image can be overlaid on a background image. The color of the image could be any color. The image could have motion included in it (the measurement reference must be stationary). The central image could be overlaid on the actual environment using the see-through screen (as in augmented reality devices). The central image can be static while the other images are controlled for the alignment, alternatively, in another embodiment of the invention the other image would be static, and the central image would be controlled for the alignment.

The measured system could be the human eye. The measured system could be an optical system with an array sensor (e.g. CCD or CMOS camera).

The cross alignment could be done simultaneously for the image as a whole, alternatively, in another embodiment of the invention, the two lines comprising the cross could be moved independently.

The collection of recorded locations could be used to fit the data to the Zernike polynomials or any other representation that could yield useful information about the measured system (e.g. Fourier series/transform).

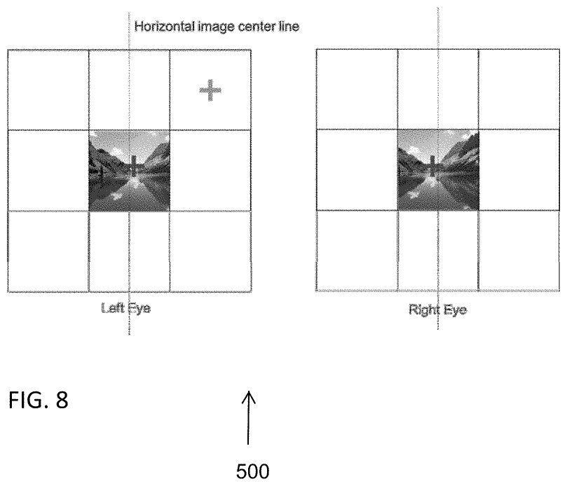

FIG. 8 depicts a central image system to create a 3D implementation for accommodation

Benefits of the Invention

Ease of Use

The optical design of the device allows for a relatively large FoV (no slits or other restricting components).

Industrial design will allow for ease of control and intuitive interaction (graphical and voice commands, UI and controls).

Speed

This measurement is relatively simple. Furthermore, the use of 2 dimensional marks allows the user to essentially perform two measurements at once, thus reducing the total number of required measurements. The user mental focus and ability to align in two dimensions is expected to be similar to that of one-dimensional alignment.

The device has no moving parts thus negating the need for wait or distraction between steps in the measurement.

Robustness

The proposed design alleviates, to some extent, the sensitivity of alignment of the measurement system with the measured system. Many of the degrees of freedom are captured in the measurement and thus are self-referenced. An example to that would be misalignment of the measured system laterally in a direction perpendicular to the optical axis of the system. This type of misalignment will be represented in a tip/tilt terms in the Zernike polynomials which are independent of the defocus and astigmatism terms for example.

Higher Order Aberration Measurement

As the measurement is based on the S-H wavefront sensor physical principles, the same rules apply as for the relation between the number of lenslets to the order of Zernike polynomials the could be represented by the measured data. Therefore, the more, lenslets and steps of measurement used, the higher the order of aberrations that can be represented.

Accommodation

Due to the static nature of the central image, in some embodiment, as well as the large FoV, it could be used as a reference image, and could be used for display as described above. Furthermore, the device could be replicated to create a binocular device. In which case, the correlation between vergence and accommodation could be used to create stereoscopic images that trigger depth perception and enable the user to direct and maintain accommodation of their eyes and vision to infinity. Thus, enabling control of the accommodation error present in the measurement. An implementation of this concept could be seen in FIG. 8.

The central segment image presented to each eye is shifted to allow for placing the measurement mark at a very far distance. Moreover, a real image, e.g. landscape, mountains, fields, could be used to further enhance the user depth perception. In an alternative embodiment, the marks are overlaid on the real environment using a see-through screen. At this case, the user would be requested to look at a far object. This has the advantage of real life, familiar, accommodation target which might improve the depth perception and thus the accommodation. The other image marks are only presented to the measured eye to enable monocular measurement for accounting for the different in refraction between the eyes.

Alternative Constructions

The device can be built such that the display could be replaced by a camera to allow for retinal imaging.

The baffles in the device, are used to prevent cross-talk between segments of the display. This can also be achieved by use of filters (each lens has a different filter) and different colors on the display (each mark/cross has a different color corresponding to the lens in front of it).

What is claimed is:

1. An optical device, comprising:
a. a lenslet array, the lenslet array having a baffle disposed between each lens; and
b. a de-magnifier comprising a first lens disposed in a first position (f1) and a second lens disposed in a second position (f2) with the distance between the first lens and second lens known as (d).

2. The system of claim 1 wherein a magnification between a display image H and an output image of the optical device h is M=h/H, with M being the magnification of the de-magnifier.

3. The system of claim 2 wherein the magnification of the de-magnifier is used to enhance the resolution of the output image.

4. The system of claim 1 wherein each lens of the lenslet array may accept a segment of a view, wherein each segmented view is presented by a display screen.

5. The system of claim 4 wherein a center lens of the lenslet array accepts a static reference image from the display screen and wherein a different lens of the lenslet array accepts a test image from the display screen and wherein alignment of the test image to the static image produces two dimensions of measured movement.

6. The system of claim 5 wherein the two dimensions of measured movement are fitted to Zernike polynomials to derive defocus and astigmatism values of a measured system.

7. The system of claim 6 wherein a plurality of two dimensional values are obtained by using a plurality of test images aligned to the static image and the plurality of two dimensional values are fitted to Zernike polynomials to derive defocus and astigmatism values of the measured system.

8. The system of claim 1 compressing two optical devices used a binocular device.

9. The system of claim 8 used to generate values of accommodation of a measured system.

10. The system of claim 8 used to present a three dimensional/stereoscopic image to a measured system to measure or induce accommodation.

11. The system of claim 1 wherein the optical device is used with a camera to facilitate retinal imaging.

12. The system of claim 2 using a display screen of a smartphone to produce the display image.

13. The system of claim 2 using an integrated display screen to produce the display image.

14. The system of claim 1 using a see through display to measure accommodation of a measured system.

15. The system of claim 1 using filters in place of baffles, the filters used to prevent cross talk between display segments.

16. A method for measuring optical distortion of a measured system, the method comprising the steps of:
a) using an optical device to present a static image and a test image to the measured system;
b) the measured system moving the test image to the static image with the movement data fitted to Zernike polynomials to derive defocus and astigmatism values of the measured system.

17. The method of claim 16 further comprising the step of
a) the optical device accepting segmented views from a display and the optical device using a lenslet array, the lenslet array comprising a plurality of lenses with the lenslet array having a baffle disposed between each lens;
b) using a de-magnifier to enhance resolution of the images received by the measured system.

18. The method of claim 17 further comprising the step of using filters in place of baffles, the filters used to prevent cross-talk between the segmented views.
